(19) United States
(12) Patent Application Publication
(10) Pub. No.: US 2005/0124916 A1
(43) Pub. Date: Jun. 9, 2005

(54) Mobile terminal having a skin permeation device, and apparatus and method for analyzing a body fluid using the same

(76) Inventors: Jong-youn Lee, Yongin-si (KR); Sang-joon Han, Seoul (KR); Kye-jin Jeon, Suwon-si (KR); In-duk Hwang, Suwon-si (KR); Hye-jin Jung, Seoul (KR)

Correspondence Address: LEE & STERBA, P.C., Suite 2000, 1101 Wilson Boulevard, Arlington, VA, 22209, US

(21) Appl. No.: 11/002,306

(22) Filed: Dec. 3, 2004

(30) Foreign Application Priority Data
Dec. 9, 2003 (KR) 2003-89079

(51) Int. Cl. A61B 17/20; A61B 5/00; B65D 81/00; C02F 1/28; B01D 15/08

(52) U.S. Cl. 600/573; 604/22; 600/584

(57) ABSTRACT

In a mobile terminal having a skin permeation device, and an apparatus and a method for analyzing a body fluid using the same, the skin permeation device includes a control unit for outputting an oscillation start signal in response to an input signal, an oscillating unit for receiving the oscillation start signal and for outputting an oscillation signal having a predetermined frequency, and an output unit for outputting an ultrasonic signal to a diagnosis region of a user in response to the oscillation signal, the output unit being operable to perform skin permeation.

### BACKGROUND OF THE INVENTION

1. Field of the Invention

The present invention relates to a mobile terminal having a skin permeation device, and an apparatus and method for analyzing a body fluid using the same. More particularly, the present invention relates to a skin permeation device using ultrasonic waves, and an apparatus and method for analyzing a body fluid extracted using the same.

2. Description of the Related Art

Diabetes is a chronic disease requiring systematic treatment. People with type 1 diabetes require more than one insulin shot a day. Insulin in the body controls a sugar concentration in blood and can prevent high blood sugar. Improper insulin treatment, however, can cause high blood sugar, which can lead to a comatose state and even death. High blood sugar can also cause heart disease, blindness, high blood pressure, and kidney failure, among other conditions. People with type 1 diabetes have to regularly check their blood sugar levels in an effort to prevent or minimize complications related to diabetes. People with type 2 diabetes can control their blood sugar levels through regulating their diet and exercising.

Generally, blood sugar concentration is measured by electrochemically analyzing blood sugar obtained by collecting blood from a patient. People with type 1 diabetes have their blood sugar concentration measured several times a day to accurately manage their blood sugar levels.

In order to prevent complications associated with diabetes, it is imperative to accurately manage blood sugar levels. However, patients sometimes avoid a periodical check of blood sugar concentration because of pain and inconvenience of the collection of blood. Therefore, in an effort to eliminate pain and improve convenience, there is a need for a mobile apparatus and method for analyzing a body fluid, such as measuring a blood sugar concentration, wherein the body fluid is collected without collecting blood.

### SUMMARY OF THE INVENTION

The present invention is therefore directed to a mobile terminal having a skin permeation device, and an apparatus and method for analyzing a body fluid using the same, which substantially overcome one or more of the problems due to the limitations and disadvantages of the related art.

It is a feature of an embodiment of the present invention to provide a mobile terminal having a skin permeation device that generates ultrasonic waves using a piezoelectric device, and an apparatus and method for analyzing a body fluid extracted using the device.

It is another feature of an embodiment of the present invention to provide a mobile terminal having a skin permeation device, and an apparatus and method for analyzing a body fluid using the same that is capable of eliminating pain and improving convenience of analyzing a body fluid.

According to an aspect of the present invention, there is provided a mobile terminal having a skin permeation device, wherein the skin permeation device includes a control unit for outputting an oscillation start signal in response to an input signal, an oscillating unit for receiving the oscillation start signal and for outputting an oscillation signal having a predetermined frequency, and an output unit for outputting an ultrasonic signal to a diagnosis region of a user in response to the oscillation signal, the output unit being operable to perform skin permeation.

The output unit may be a piezoelectric device.

The predetermined frequency may be a resonant frequency of a piezoelectric device.

The output unit may be integrated with the mobile terminal and may be mounted on a portion of the mobile terminal that can contact the diagnosis region. Alternatively, the output unit may be a separate apparatus and may be wired to the oscillating unit in the mobile terminal to receive the oscillation signal.

The skin permeation device may be operable to inject a medicine into the user or apply cosmetics to the user.

According to another aspect of the present invention, there is provided an apparatus for analyzing a body fluid including a mobile terminal for outputting an ultrasonic wave to a diagnosis region of a user to perform skin permeation and a sensor for analyzing the body fluid extracted from the diagnosis region by the skin permeation.

The mobile terminal may include a control unit for outputting an oscillation start signal in response to an input signal, an oscillating unit for receiving the oscillation start signal and for outputting an oscillation signal having a predetermined frequency, and an output unit for outputting an ultrasonic signal to a diagnosis region of a user in response to the oscillation signal, the output unit being operable to perform skin permeation.

The output unit may be a piezoelectric device.

The output unit may be integrated with the mobile terminal and may be mounted on a portion of the mobile terminal that can contact the diagnosis region. Alternatively, the output unit may be a separate apparatus and may be wired to the oscillating unit in the mobile terminal to receive the oscillation signal.

The sensor may further include a transmitting unit for outputting a result of the analysis of the body fluid to the mobile terminal, the mobile terminal may further include a receiving unit for receiving the result of the analysis of the body fluid.

The sensor may further include a body fluid detecting unit for detecting an amount of body fluid extracted and for outputting the detected amount to the receiving unit through the transmitting unit, wherein the control unit is operable to determine whether the detected amount of the body fluid is sufficient for purposes of analysis and to continue transmission of the oscillation start signal or to output a signal to end the oscillation to the oscillating unit depending on the determined result.

The body fluid detecting unit may be operable to sense an amount of an indicating material used in the analysis of the body fluid that reacts with the body fluid and may be operable to detect the amount of the body fluid.

The mobile terminal may further include a storage unit for temporarily receiving a result of skin permeation from the receiving unit and for storing the result when a telephone call is answered during analysis of the body fluid. The mobile terminal may further include a display unit for displaying a result of the skin permeation to a user.

According to still another aspect of the present invention, there is provided a method of analyzing a body fluid including switching a mode of a mobile terminal to a mode for analyzing the body fluid, attaching a sensor for analyzing the body fluid to a diagnosis region, generating and applying ultrasonic waves to the diagnosis region, and extracting the body fluid from the diagnosis region, detecting an amount of body fluid extracted and outputting the detected amount to the mobile terminal, determining whether the amount of body fluid extracted is sufficient to analyze the body fluid, and ending generation of the ultrasonic waves when the amount of body fluid extracted is sufficient, and analyzing the body fluid.

The mobile terminal may extract additional body fluid until the amount of body fluid extracted is sufficient for analysis by continuing to generate the ultrasonic waves if the amount of body fluid extracted is insufficient for analysis.

Detecting the amount of body fluid extracted may include measuring an amount of an indicating material used in the analysis of the body fluid that reacts with the body fluid.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1A illustrates an exemplary mobile terminal according to an embodiment of the present invention having an ultrasonic output unit that is exposed when the mobile terminal is unfolded;

FIG. 1B illustrates another exemplary mobile terminal according to another embodiment of the present invention having an ultrasonic output unit that is exposed when the mobile terminal is folded;

FIG. 1C illustrates still another exemplary mobile terminal according to still another embodiment of the present invention having a separate ultrasonic output unit that is wired to the mobile terminal;

FIG. 2 illustrates a user using a mobile terminal having a skin permeation device according to an embodiment of the present invention;

FIG. 3 is a block diagram of a skin permeation device provided in a mobile terminal according to an embodiment of the present invention;

FIG. 4 is a graph of skin permeation degree versus a pressing difference amplitude of a piezoelectric device;

FIG. 5 is a block diagram of a sensor in a skin permeation device according to an embodiment of the present invention; and

FIG. 6 is a flow chart of a method of analyzing a body fluid according to an embodiment of the present invention.

### DETAILED DESCRIPTION OF THE INVENTION

Korean Patent Application No. 2003-89079, filed on Dec. 9, 2003, in the Korean Intellectual Property Office, and entitled: “Mobile Terminal Including a Skin Permeation Device, and Apparatus and Method for Analyzing a Body Fluid Using the Same,” is incorporated by reference herein in its entirety.

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals and characters indicate like elements throughout.

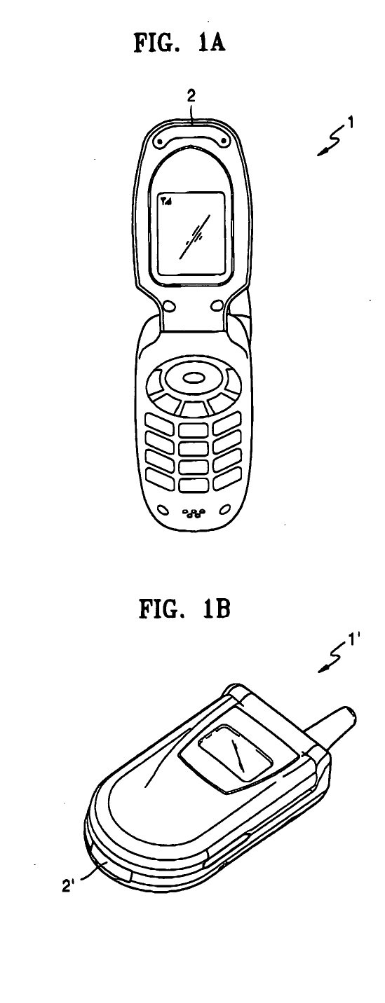

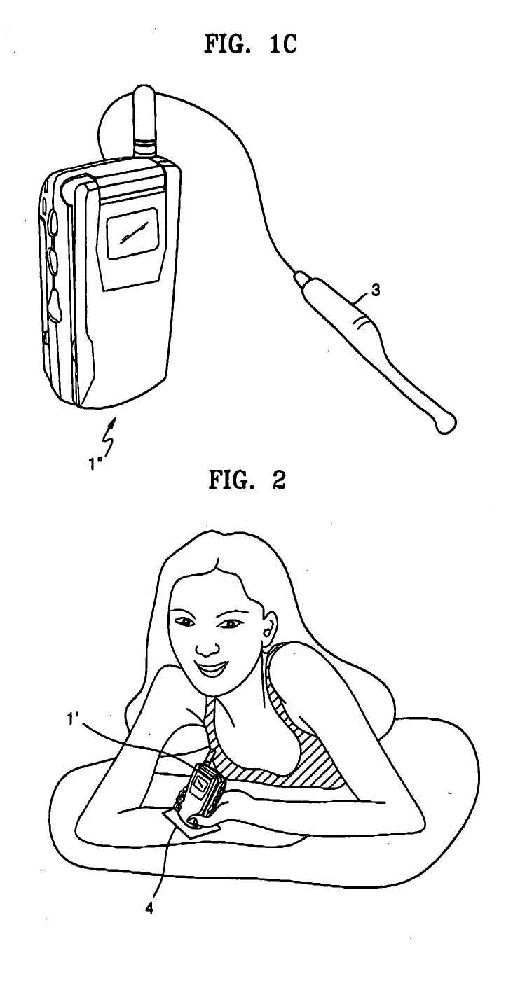

FIGS. 1A through 1C illustrate an external appearance of various exemplary mobile terminals having a skin permeation device according to various embodiments of the present invention. FIG. 1A illustrates an exemplary mobile terminal 1 according to an embodiment of the present invention having an integrated ultrasonic output unit 2 that is mounted on a portion of the mobile terminal 1 that may contact a diagnosis region of a user, the ultrasonic output unit 2 being exposed when the exemplary mobile terminal 1 is unfolded. FIG. 1B illustrates another exemplary mobile terminal 1′ according to another embodiment of the present invention having an integrated ultrasonic output unit 2′ that is mounted on a portion of the mobile terminal 1 that may contact a diagnosis region of a user, the ultrasonic output unit 2′ being exposed when the exemplary mobile terminal 1 is folded. FIG. 1C illustrates still another exemplary mobile terminal 1″ according to still another embodiment of the present invention having a separate ultrasonic output unit 3 that may contact a diagnosis region of a user, the ultrasonic output unit 3 being wired to the exemplary mobile terminal 1″. Each of the exemplary mobile terminals 1, 1′, or 1″ is portable and may be embodied as a mobile phone, as illustrated, as a personal digital assistant (PDA), or other similar device.

FIG. 2 illustrates a user using a mobile terminal having a skin permeation device according to an embodiment of the present invention. The user performs skin permeation by contacting the ultrasonic output unit 2′ to a diagnosis region on the user's body. A sensor 4 is provided in connection with the skin permeation device to confirm whether a body fluid is extracted during the skin permeation and to analyze the extracted body fluid. The sensor 4 will be described in greater detail below.

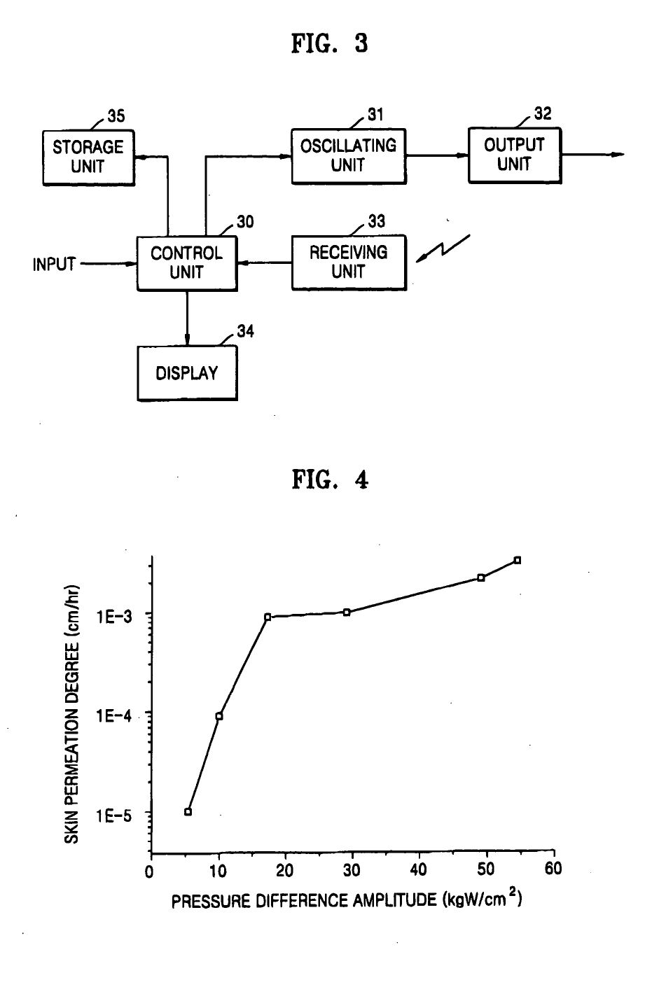

FIG. 3 is a block diagram of a skin permeation device provided in a mobile terminal according to an embodiment of the present invention. The skin permeation device includes a control unit 30, an oscillating unit 31, and an output unit 32. The output unit 32 is an ultrasonic output unit according to an embodiment of the present invention, and may be a piezoelectric device, e.g., a piezoelectric transducer.

The skin permeation device may further include a receiving unit 33 for receiving a signal of skin permeation degree from the sensor 4 applied to the user's skin. In addition, the skin permeation device may include a storage unit 35 for storing a result of the skin permeation or a display 34 for displaying a result of the skin permeation to the user.

The storage unit 35 may act as a buffer. For example, if the mobile terminal is a phone and the user answers a call and talks over the phone while in the process of analyzing a body fluid using the mobile terminal 1, 1′, or 1″, a signal of the degree of skin permeation received from the sensor 4 is temporarily stored in the storage unit 35. When the call is terminated, the control unit 30 may inform the user of the temporarily stored data and continue to analyze the body fluid.

The control unit 30 outputs an oscillation start signal to the oscillating unit 31, when an input signal for skin permeation is received. The input signal may be input using voice recognition or by pushing a button for skin permeation separately provided on the mobile terminal 1, 1′, or 1″.

The oscillating unit 31 outputs an oscillation signal to the output unit 32 according to the oscillation start signal. If the output unit 32 is a piezoelectric device, a frequency of the oscillation signal may be a resonant frequency of a piezoelectric device. The output unit 32 generates an ultrasonic signal in response to the oscillation signal.

Typically, a mobile terminal includes a piezoelectric device for generating sound waves in an audio frequency band of between about 20 Hz-20 kHz. The ultrasonic frequency band used for skin permeation is between about 10 kHz-1 MHz and the intensity is about 1-7 Watt/cm².

Accordingly, the piezoelectric device, which is used for generating sounds in a mobile terminal, may be employed as the output unit 32 to generate a necessary ultrasonic wave. In this case, the frequency and intensity require consideration. A problem of the ultrasonic frequency band may be solved because the piezoelectric device implemented in a typical mobile terminal is able to generate a frequency of over 20 kHz. However, the intensity generated from the piezoelectric device implemented in a typical mobile terminal is only 0.1 Watt/cm², which needs to be increased.

Typically, an intensity I of a piezoelectric device may be expressed by Equation (1) as follows:

$$I = \frac{{P}^{2}}{2 ρ C} , \qquad ( 1 )$$

where P is a range of change in pressure applied to the piezoelectric device in units of kgW/cm², ρ is a density of a medium in units of kg/cm³, and C is a speed of an ultrasonic wave in units of cm/sec.

FIG. 4 is a graph of skin permeation degree versus a pressing difference amplitude of a piezoelectric device.

The skin permeation result shown in FIG. 4 may be obtained by calculating skin permeation according to Equation (1) while adjusting the range of change in pressure applied to the piezoelectric device with a frequency of 20 kHz, assuming that skin density is 0.001 kg/cm³ and the speed of an ultrasonic wave in skin tissue is 1487 cm/sec. Data on skin density, speed of an ultrasonic wave in skin tissue, and other related data have been disclosed by W. S. Snyder, et al. in a publication entitled: “Report of the Task Group on Reference Man,” Oxford, UK, Pergamon, 1975.

Typically, skin should be permeated at a rate greater than 10⁻³ cm/hr in order to extract ions or inject medicine. As shown in FIG. 4, it is possible to generate an ultrasonic wave from a mobile terminal when adopting a device generating a pressing difference amplitude of about 17.2 kgW/cm² in the mobile terminal. Since large intensity ultrasonic waves generate heat, the pressing difference amplitude may be adjusted within a range not to exceed about 54.4 kgW/cm².

In operation, the receiving unit 33 receives a signal indicating a degree of skin permeation output from the sensor 4 and outputs how much body fluid has been extracted. The output value may be an amount of body fluid extracted or a result of the body fluid analysis obtained from the sensor 4. The result of the analysis may be a digitized value of a reaction degree of an indicating material included in the sensor 4 to analyze the body fluid. That is, when the body fluid is analyzed using the indicating material reacting to the body fluid, the result of the analysis may be obtained by sensing an amount of the indicating material reacted with the body fluid.

The control unit 30 determines whether the amount of the body fluid output from the receiving unit 33 is sufficient to be analyzed. If the amount of the body fluid is insufficient, the control unit 30 continues transmission of the oscillation start signal to the oscillating unit 31. If the amount of the body fluid is sufficient, the control unit 30 outputs a signal to the oscillating unit 31 to end the oscillation. The amount of the body fluid sufficient for analysis can vary depending on a purpose or method of the analysis. The control unit 30 displays a finally determined result of the analysis.

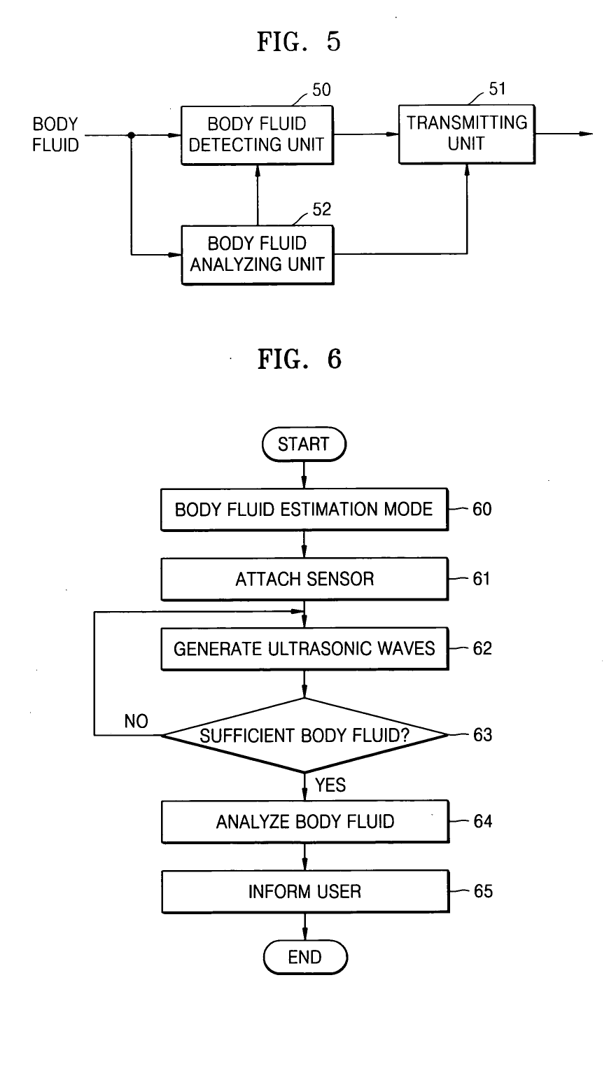

FIG. 5 is a block diagram of the sensor 4 in the skin permeation device according to an embodiment of the present invention. The sensor 4 may be mounted on the diagnosis region of the user or may be patched onto the diagnosis region using MEMS technology.

When body fluid is extracted from hypodermal tissues through skin permeation, a body fluid detecting unit 50 determines whether the amount of the body fluid extracted is sufficient to be analyzed. The determination may be achieved by a scheme of applying an ultrasonic wave for a predetermined time period, which is obtained experimentally by measuring the extracted amount via a sensor (not shown), or by measuring a value related to the analysis performed by a body fluid analyzing unit 52.

The body fluid analyzing unit 52 may measure a concentration of a component in the body fluid, such as blood sugar, hemoglobin, a degree of oxygen saturation, bilirubin, glucose, and the like, by including a medium containing an indicating material causing a chemical reaction with interstitial fluid that exist between cells or in the area surrounding cells or by including a medium for performing ion separation of electrolytes from the body fluid.

The body fluid detecting unit 50 outputs the measured amount of the body fluid or a result value of the body fluid analysis received from the body fluid analyzing unit 52 to a transmitting unit 51. The transmitting unit 51 further outputs the results to the receiving unit 33 of FIG. 3.

FIG. 6 is a flow chart of a method of analyzing a body fluid according to an embodiment of the present invention.

In operation 60, a user switches the mobile terminal 1, 1′, or 1″ to a body fluid measurement mode to analyze a body fluid. After switching the mode of the mobile terminal 1, 1′, or 1″, in operation 61, the user attaches the sensor 4 to the diagnosis region. In operation 62, an ultrasonic wave is generated by contacting the output unit 32 of the mobile terminal 1 to the sensor 4. The order of the sensor application operation 61 and the ultrasonic wave generation operation 62 may be reversed. That is, after the sensor 4 is initially applied to the diagnosis region and then the ultrasonic wave is generated onto the sensor 4, the body fluid may be extracted, or after the ultrasonic wave is generated, the sensor 4 may be applied to the diagnosis region from which the body fluid is extracted.

When the body fluid is extracted, the body fluid detecting unit 50 outputs information on the amount of body fluid extracted to the control unit 30. In operation 63, it is determined whether a sufficient amount of body fluid has been extracted. If the amount of the body fluid is insufficient for analysis purposes, the control unit 30 repeats operation 62 to extract additional body fluid. If the amount is sufficient, the control unit 30 stops the generation of ultrasonic waves. In operation 64, the body fluid analyzing unit 52 then analyzes the extracted body fluid and outputs the analyzed values, e.g., blood sugar concentration, through the transmitting unit 51. In operation 65, the control unit 30 informs the user of the results of the analysis when the analyzed values are determined to be final.

When skin permeation by the skin permeation device according to the present invention is compared with skin permeation performed conventional electrophoresis, time-lapse is greatly reduced from two hours to about sixteen seconds. Also, once performed, the skin permeation by ultrasonic wave can continue for twenty-four hours whereas the skin permeation by electrophoresis can continue for only thirteen hours. Thus, the present invention can guarantee a relatively long successive measurement time.

Skin permeation may be used not only for analyzing body fluid, but also for injecting a medicine. As opposed to oral administration of a medication, skin permeation can avoid metabolism in the digestive organs, maintain an effect of the medication for more than a week, and avoid an effect that most of the medicine is absorbed by the liver or the stomach before transmission of the medicine by body circulation. As opposed to administration of a medication by needle injection, administration of a medication by skin permeation eliminates risks of infection and pain, maintains a regular dosage of medicine, and is theoretically superior in the transmission of protein for medical treatment. Further, skin permeation can also be applied to an application of cosmetics.

According to an embodiment of the present invention, it is possible to extract a body fluid without collecting blood, e.g., using an injection needle, at any time and place using a mobile terminal having a skin permeation device. Moreover, user's mobility may not be impeded by implementing a patch-type sensor to analyze extracted body fluid.

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

1. A mobile terminal comprising a skin permeation device, wherein the skin permeation device includes:
a control unit for outputting an oscillation start signal in response to an input signal;
an oscillating unit for receiving the oscillation start signal and for outputting an oscillation signal having a predetermined frequency; and
an output unit for outputting an ultrasonic signal to a diagnosis region of a user in response to the oscillation signal, the output unit being operable to perform skin permeation.

2. The mobile terminal as claimed in claim 1, wherein the output unit is a piezoelectric device.

3. The mobile terminal as claimed in claim 2, wherein the predetermined frequency is a resonant frequency of the piezoelectric device.

4. The mobile terminal as claimed in claim 1, wherein the output unit is integrated with the mobile terminal and is mounted on a portion of the mobile terminal that can contact the diagnosis region.

5. The mobile terminal as claimed in claim 1, wherein the output unit is a separate apparatus and is wired to the oscillating unit in the mobile terminal to receive the oscillation signal.

6. The mobile terminal as claimed in claim 1, wherein the skin permeation device is operable to inject a medicine into the user.

7. The mobile terminal as claimed in claim 1, wherein the skin permeation device is operable to apply cosmetics to the user.

8. An apparatus for analyzing a body fluid, comprising:
a mobile terminal for outputting an ultrasonic wave to a diagnosis region of a user to perform skin permeation; and
a sensor for analyzing the body fluid extracted from the diagnosis region by the skin permeation.

9. The apparatus as claimed in claim 8, wherein the mobile terminal comprises:
a control unit for outputting an oscillation start signal in response to an input signal;
an oscillating unit for receiving the oscillation start signal and for outputting an oscillation signal having a predetermined frequency; and
an output unit for outputting an ultrasonic signal to a diagnosis region of a user in response to the oscillation signal, the output unit being operable to perform skin permeation.

10. The apparatus as claimed in claim 9, wherein the output unit is a piezoelectric device.

11. The apparatus as claimed in claim 9, wherein the output unit is integrated with the mobile terminal and is mounted on a portion of the mobile terminal that can contact the diagnosis region.

12. The apparatus as claimed in claim 9, wherein the output unit is a separate apparatus and is wired to the oscillating unit in the mobile terminal to receive the oscillation signal.

13. The apparatus as claimed in claim 9, wherein the sensor further comprises a transmitting unit for outputting a result of the analysis of the body fluid to the mobile terminal, the mobile terminal further including a receiving unit for receiving the result of the analysis of the body fluid.

14. The apparatus as claimed in claim 13, the sensor further comprising:
a body fluid detecting unit for detecting an amount of body fluid extracted and for outputting the detected amount to the receiving unit through the transmitting unit,
wherein the control unit is operable to determine whether the detected amount of the body fluid is sufficient for purposes of analysis and to continue transmission of the oscillation start signal or to output a signal to end the oscillation to the oscillating unit depending on the determined result.

15. The apparatus as claimed in claim 14, wherein the body fluid detecting unit is operable to sense an amount of an indicating material used in the analysis of the body fluid that reacts with the body fluid and is operable to detect the amount of the body fluid.

16. The apparatus as claimed in claim 13, wherein the mobile terminal further comprises a storage unit for temporarily receiving a result of skin permeation from the receiving unit and for storing the result when a telephone call is answered during analysis of the body fluid.

17. The apparatus as claimed in claim 13, wherein the mobile terminal further comprises a display unit for displaying a result of the skin permeation to a user.

18. A method of analyzing a body fluid, comprising:
switching a mode of a mobile terminal to a mode for analyzing the body fluid;
attaching a sensor for analyzing the body fluid to a diagnosis region, generating and applying ultrasonic waves to the diagnosis region, and extracting the body fluid from the diagnosis region;
detecting an amount of body fluid extracted and outputting the detected amount to the mobile terminal;
determining whether the amount of body fluid extracted is sufficient to analyze the body fluid, and ending generation of the ultrasonic waves when the amount of body fluid extracted is sufficient; and
analyzing the body fluid.

19. The method as claimed in claim 18, wherein the mobile terminal extracts additional body fluid until the amount of body fluid extracted is sufficient for analysis by continuing to generate the ultrasonic waves if the amount of body fluid extracted is insufficient for analysis.

20. The method as claimed in claim 18, wherein detecting the amount of body fluid extracted comprises measuring an amount of an indicating material used in the analysis of the body fluid that reacts with the body fluid.
